(19) United States
(12) Patent Application Publication
(10) Pub. No.: US 2021/0361868 A1
(43) Pub. Date: Nov. 25, 2021

(54) Guiding Musculoskeletal Procedures

(71) Applicant: Agitated Solutions Inc., Oakdale, MN (US)

(72) Inventors: Morgan Evans, Apple Valley, MN (US); Kenneth Trauner, San Francisco, CA (US)

(73) Assignee: Agitated Solutions Inc., Oakdale, MN (US)

(21) Appl. No.: 17/322,061

(22) Filed: May 17, 2021

Related U.S. Application Data
(60) Provisional application No. 63/026,198, filed on May 18, 2020.

(51) Int. Cl. A61M 5/19; A61M 5/00; A61K 31/573; A61K 31/167; A61K 9/00; A61K 49/22

(52) U.S. Cl. CPC A61M 5/19; A61M 5/007; A61K 31/573; A61M 2202/048; A61K 9/0019; A61K 49/223; A61K 31/167

(57) ABSTRACT

A method may include providing a syringe device having (a) a single needle, (b) a first syringe comprising saline with microbubbles, (c) a second syringe comprising a therapeutic compound, and (d) a connector coupling the single needle to the first syringe and to the second syringe. The method may further include guiding the single needle to an injection site of a patient; injecting, with the first syringe, a quantity of saline with microbubbles; confirming a desired position of the single needle, with ultrasound imaging, by identifying the microbubbles in the quantity of saline relative to a tip of the single needle and further relative to one or more anatomical landmarks visible with the ultrasound imaging; and injecting, with the second syringe, the therapeutic compound at the injection site.

### CROSS-REFERENCE TO RELATED APPLICATIONS

This application claims the benefit of U.S. Provisional Application Ser. No. 63/026,198, titled “Guiding Musculoskeletal Injections,” filed on May 18, 2020. This application incorporates the entire contents of the foregoing application herein by reference.

### TECHNICAL FIELD

Various embodiments relate generally to providing relief for musculoskeletal conditions.

### BACKGROUND

Joint aspiration is a procedure to remove fluid from the space around a joint. It is typically performed using a needle and a syringe, often under local anesthetic, to relieve swelling or to obtain fluid for analysis, to diagnose joint orders or issues. Joint aspiration is most commonly performed on a knee, but it may also be performed on a hip, ankle, shoulder, elbow or wrist. In some instances, an injection may be provided following the joint aspiration; in other instances, an injection in or around a joint may be provided as a stand-alone therapy for a joint issue. Corticosteroids (e.g., hydrocortisone, methylprednisolone, triamcinolone, dexamethasone), local anesthetics (e.g., lidocaine, bupivacaine), saline, hyaluronic acid, or other compounds may be injected to provide relief from joint issues.

### SUMMARY

In some implementations, a method includes providing a syringe device having (a) a single needle, (b) a first syringe comprising saline with microbubbles, (c) a second syringe comprising a therapeutic compound, and (d) a connector coupling the single needle to the first syringe and to the second syringe. The method may further include guiding the single needle to an injection site of a patient; injecting, with the first syringe, a quantity of saline with microbubbles; confirming a desired position of the single needle, with ultrasound imaging, by identifying the microbubbles in the quantity of saline relative to a tip of the single needle and further relative to one or more anatomical landmarks visible with the ultrasound imaging; and injecting, with the second syringe, the therapeutic compound at the injection site.

In some implementations, the therapeutic compound comprises a corticosteroid. In some implementations, the therapeutic compound comprises lidocaine.

In some implementations, a method includes providing a syringe device having (a) a single needle, (b) a first syringe comprising saline with microbubbles, (c) a second syringe configured to aspirate fluid from a treatment site of a patient, and (d) a connector coupling the single needle to the first syringe and to the second syringe. The method may further include guiding the single needle to the treatment site; injecting, with the first syringe, a quantity of saline with microbubbles; confirming a desired position of the single needle, with ultrasound imaging, by identifying the microbubbles in the quantity of saline relative to a tip of the single needle and further relative to one or more anatomical landmarks visible with the ultrasound imaging; and aspirating, with the second syringe, fluid from the treatment site.

In some implementations, a method includes providing a syringe device having (a) a single needle, (b) a first syringe comprising saline with microbubbles and having a first-syringe plunger, (c) a second syringe having a second-syringe plunger that is independently operable relative to the first-syringe plunger, and (d) a connector coupling the single needle to the first syringe and to the second syringe. The method may further include guiding the single needle to a treatment site of a patient; injecting, with the first syringe, a quantity of saline with microbubbles; confirming a desired position of the single needle, with ultrasound imaging, by identifying the microbubbles in the quantity of saline relative to a tip of the single needle and further relative to one or more anatomical landmarks visible with the ultrasound imaging; and with the second syringe, injecting a therapeutic compound at the treatment site or aspirating fluid from the treatment site.

Injecting with the first syringe may include depressing the first-syringe plunger. The second syringe may include a therapeutic compound, and injecting the therapeutic compound may include depressing the second-syringe plunger. The second syringe may be configured to aspirate fluid from a treatment site of a patient, and aspirating fluid from the treatment site may include drawing back the second-syringe plunger.

### BRIEF DESCRIPTION OF THE DRAWINGS

FIG. 1A depicts an exemplary injection into a subacromial bursa.

FIG. 1B depicts an exemplary injection into a glenohumeral joint.

FIG. 1C illustrates exemplary structures adjacent a subacromial bursa and glenohumeral joint.

FIG. 2A depicts an exemplary ultrasound-guided procedure.

FIG. 2B depicts an exemplary ultrasound-guided procedure with bubbles.

FIG. 3 illustrates an exemplary needle and syringe device.

FIG. 4 illustrates an exemplary method for guiding injections.

FIG. 5 illustrates another exemplary needle and syringe device.

FIG. 6 illustrates an exemplary method for performing an aspiration.

### DETAILED DESCRIPTION

A variety of injuries and conditions can affect joints, causing swelling, pain, loss of mobility, and other issues. For many of these conditions, aspiration of joint fluid may be indicated; an injection of a local anesthetic, corticosteroid or other compound may provide relief; or some other orthopedic or musculoskeletal procedure may be indicated.

Aspirations and injections are both typically performed with needles and syringes. Because of the complex structures that surround most joints (skeletal structures, vessels, nerves, bursa, ligaments, tendons, etc.), it is critical that any needle used to aspirate or deliver a therapeutic compound is properly placed relative to the surrounding structures.

Failure to properly position a needle or deliver a corticosteroid or other compound to other than an intended space or structure can have very serious effects. For example, in some cases, misapplication of a corticosteroid can result in neuritis (minor irritation to nerves), thinning of the bones (osteoporosis), avascular necrosis (serious damage to the bones of the large joints), tissue damage or tendon rupture, septic arthritis, necrotizing fasciitis, osteomyelitis, spinal cord or peripheral nerve injuries, or other serious complications. Given the number of different joints that may be treated, and the various indications for aspiration or delivery of a corticosteroid or other compound, any tools or aids for needle positioning can lower procedure risk.

Image-based guidance may be employed in many procedures. For example, ultrasound and x-ray fluoroscopy are often employed; and in some cases, computed tomography (CT) or magnetic resonance imaging (MRI) guidance may be employed. X-ray fluoroscopy typically exposes a patient to high levels of radiation; and CT and MRI guidance require specialized equipment that can greatly limit the range of movement for both patient and physician during a procedure. Ultrasound guidance is convenient and safe—it does not employ harmful radiation, and most therapy venues have ready access to ultrasound equipment and to trained technicians to use the equipment. However, image quality may be lower with ultrasound than with other imaging modalities; and ultrasound image quality may be impacted by the precise placement of the ultrasound transducer and by the skill of the ultrasound technician.

In some procedures, agitated saline (saline with microbubbles) may significantly enhance ultrasound guidance of needles used in musculoskeletal procedures. To underscore the need and benefit of any additional guidance that may be provided, various joints and indications are enumerated; then some detail is provided regarding a shoulder joint—an exemplary complex joint with many structures that must be navigated in an aspiration or injection procedure.

There are various indications for injections in an ankle, including, as examples, osteoarthritis, rheumatoid arthritis, acute traumatic arthritis, crystalloid deposition disease, mixed connective tissue disease, and synovitis. For the elbow, exemplary indications can include tennis elbow, arthritis and bursitis. For the hip: bursitis and arthritis. For the knee: osteoarthritis and bursitis. For the shoulder: rotator cuff disease (degenerative tendonitis, impingement, partial tears and subacromial bursitis), adhesive capsulitis (“frozen shoulder”), glenohumeral osteoarthritis, acromioclavicular joint disease (osteoarthritis or osteolysis) and bursitis. For the spine: chronic back pain, lumbar facet arthropathy, sacroiliac joint pain syndrome, arthritis and spondyloarthropathy. For the wrist and hand: carpal tunnel syndrome, first carpometacarpal joint disease, De Quervain's Tenosynovitis, ganglion cysts, and Trigger Finger. For the feet: plantar fasciitis of the foot (heel spurs), Morton neuromas in the foot, gout and psoriatic arthritis.

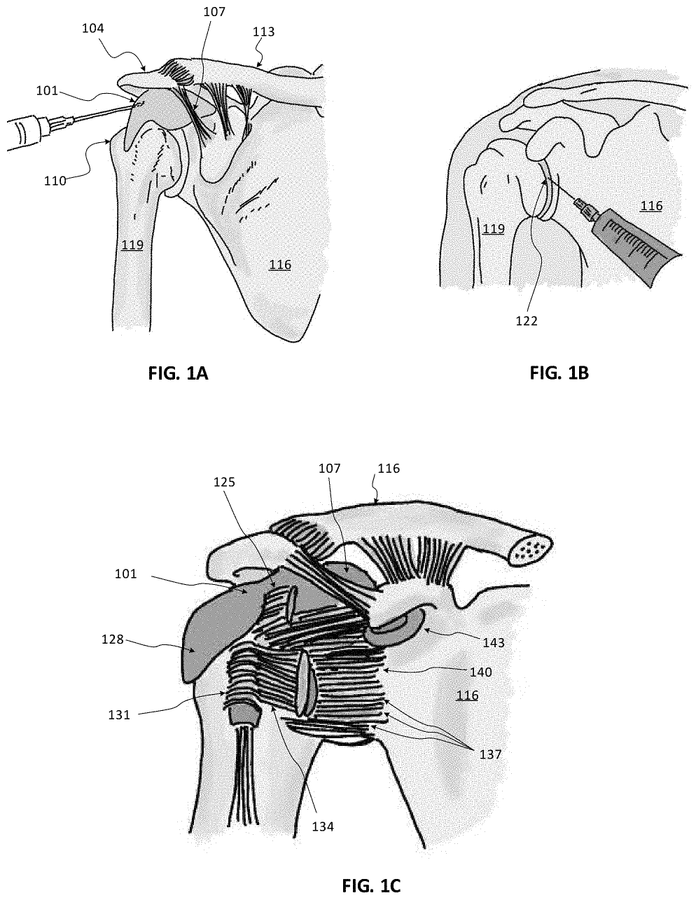

In each of these cases, aspiration or injection requires careful positioning of a needle. This point is underscored with reference to FIGS. 1A, 1B and 1C. FIG. 1A. depicts an injection in the subacromial bursa 101. For reference, this bursa 101 (a fluid-filled sac or saclike cavity that counters friction at a joint) is disposed below the acromion 104 and next to the coracoacromial ligament 107. For reference, a number of additional anatomical features are noted in FIG. 1A, including the greater tuberosity of the humerus 110, the clavicle 113, the scapula 116, and the humerus 119. FIG. 1B depicts an injection into a different shoulder structure—namely, the glenohumeral joint 122.

FIG. 1C provides additional anatomical detail around the subacromial bursa 101 and the glenohumeral joint 122—underscoring how carefully a needle must be navigated in the exemplary procedures depicted in FIG. 1A and FIG. 1B. In particular, the tendon of the supraspinatus muscle 125 (shown in partial cutaway for clarity) is anchored directly below the subacromial bursa 101; the subdeltoid bursa 128 is positioned adjacent the subacromial bursa 101; the transverse humeral ligament 131 is anchored below the subdeltoid bursa 128, and the tendon of subscapularis muscle 134 is adjacent. Over the glenohumeral joint (hidden in FIG. 1C) are the glenohumeral ligaments 137 and articular capsule 140; and nearby is the subcoracoid bursa 143.

As the reader will appreciate from FIGS. 1A, 1B and 1C., there are many ligaments, bursa, bones and tendons in very close proximity to structures of interest (e.g., the exemplary subacromial bursa 101 or glenohumeral joint 122 depicted in FIGS. 1A and 1B, respectively), around which a healthcare provider must carefully navigate a needle.

These specific structures are exemplary for the shoulder. Although a shoulder joint is complex, given its wide range of motion, elbows, wrists, knees, hips and ankles all have a similar array of ligaments, bursa, bones and tendons that must also be navigated around. The reader will appreciate that the concepts described herein can be extended to other joints and procedures.

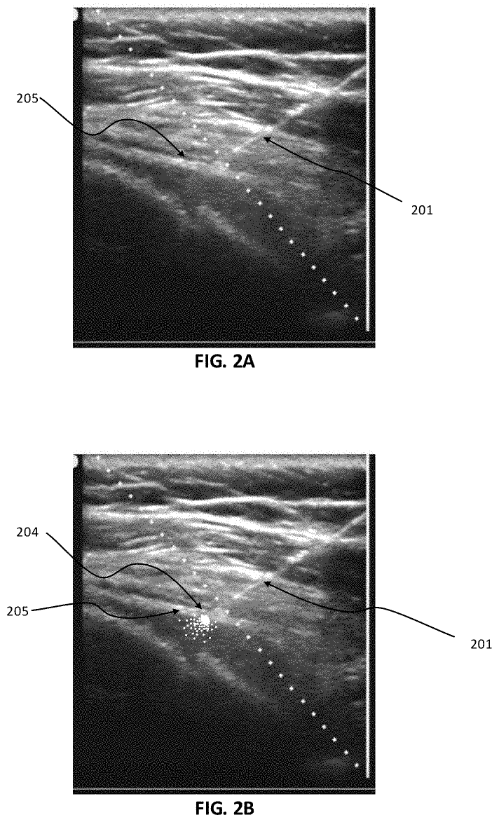

FIG. 2A illustrates exemplary ultrasound imaging that may be used to guide a needle during an injection or aspiration. As shown in the frame of FIG. 2A, various layers of tissue (muscle, ligaments, tendons, etc.) appear as contours. For example, the tissue plane 205 appears as a light-colored contour, given its hyperechogenic nature (ability to reflect ultrasound signals). In FIG. 2A, the needle 201 is partially visible, but its depth and the precise location of its tip are very difficult to identify. The reader will appreciate that even a skilled ultrasound technician must perform procedures very carefully—regularly pausing to confirm needle position by moving the ultrasound transducer and identifying anatomical landmarks on the screen. Even by doing this, the tip of the needle 201 may still be difficult to locate. If this tip is not in the desired spot when an aspiration is performed or an injection given, traumatic or catastrophic results may result (e.g., nerve damage, bone damage, tendon damage, etc.).

FIG. 2B depicts the same ultrasound video as that of FIG. 2A, but with bubbles 204 employed to identify the tip of the needle 201. As depicted, a small amount of agitated saline has been injected through the needle 201. As shown, the bubbles 204 in the injected saline show up on the ultrasound image and provide an additional reference that may enable an ultrasound technician or healthcare provider to ascertain a more precise location of the tip. In addition, the bubbles 204, relative to other anatomical structures in the ultrasound image (e.g., the tissue plane 205), provide additional detail regarding location of the needle tip.

Small bubbles (e.g., microbubbles) in saline or similar solution may be superior to simply injecting a bolus of air into a patient. Their very small size enables them to be quickly absorbed into the surrounding tissue, without causing cramping or other issues in the patient. In addition, given the dispersion of the bubbles, they may more clearly indicate the location of the tip of needle, relative to layers of tissue (e.g., the tissue layer 205), whose depth may also be more clearly indicated by the bubbles.

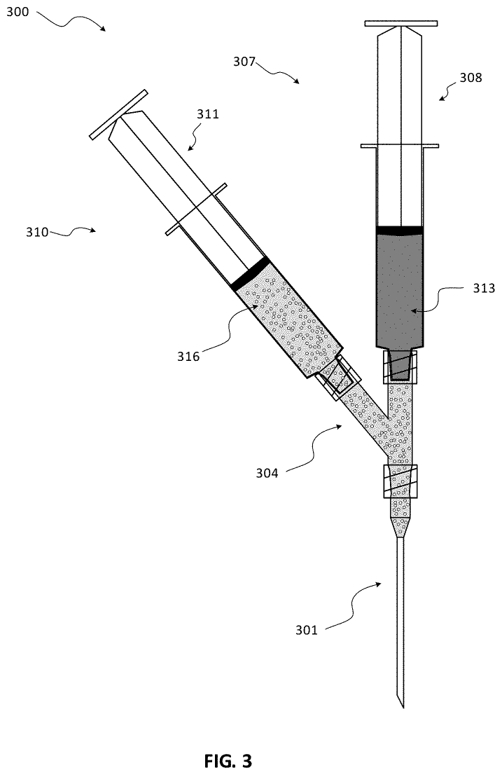

FIG. 3 illustrates an exemplary needle and syringe device 300 that can deliver both bubbles and a therapeutic compound, such as corticosteroids, to, for example, a joint. As shown, the device 300 includes a single needle 301, a Y-connector 304 (as shown, with Luer lock connectors), a first syringe 310 having a first-syringe plunger 311, and a second syringe 307 having a second-syringe plunger 308. As shown, the first-syringe plunger 311 and second-syringe plunger 308 are independently operable. The first syringe 310 may contain an agitated saline 316 for injecting a small quantity of bubbles to help a medical care provider to place the needle 301, as described with reference to FIG. 2B; and the second syringe 307 may contain a corticosteroid 313, or other therapeutic compound, for injection into a patient's joint.

The device 300 illustrated in FIG. 3 is shown having two equally sized syringes coupled by a rigid Y-connector 304, but the device 300 could take other forms. For example, the agitated saline 316 could be coupled to the Y-connector 304 with flexible tubing to make the overall device 300 more easy to manipulate with one hand by a medical care provider who may be both operating the ultrasound transducer and performing an injection. The syringes 307 and 310 may be larger or smaller or differently sized. Rather than the needle 301 having a single lumen, it may be a double-lumen needle, and each lumen may be separately connected to a different syringe. Other variations are possible.

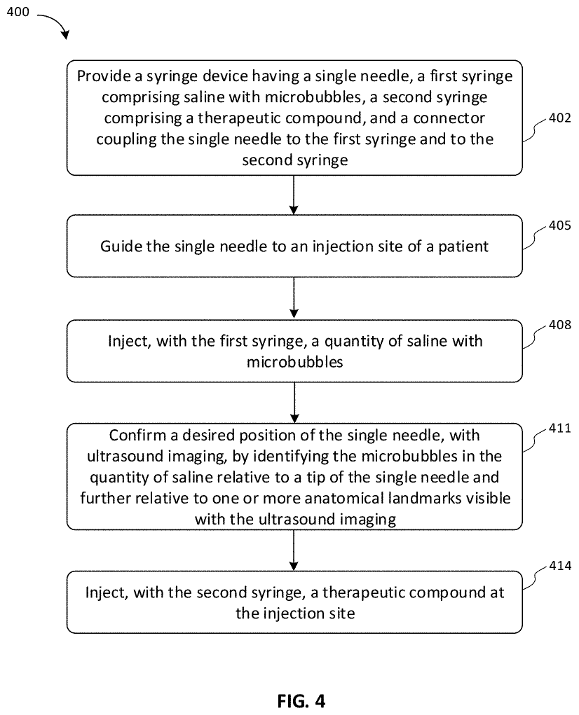

FIG. 4 illustrates an exemplary method 400 for guiding injections. As illustrated, the method 400 includes providing (402) a syringe device. In particular, the method 400 includes providing (402) a syringe device having a single needle (e.g., the device 300, shown in FIG. 3, having a single needle 301), a first syringe comprising saline with microbubbles (e.g., syringe 310), a second syringe comprising a a therapeutic compound (e.g., syringe 310), and a connector coupling the single needle to the first syringe and second syringe (e.g., connector 304).

The method 400 may include guiding (405) the single needle to an injection site of a patient. For example, with reference to FIG. 1B, the method 400 may include guiding the single needle to the glenohumeral joint of a patient.

The method 400 may include injecting (408), with the first syringe, a quantity of saline with microbubbles. For example, with reference to FIG. 3, the method 400 could include injecting (408), e.g., by depressing the first-syringe plunger 311, to inject a quantity of saline with microbubbles.

The method 400 may include confirming (411) the desired position of the single needle, with ultrasound imaging. Confirming (411) the desired position of the single needle could include identifying microbubbles in the quantity of saline (via ultrasound imaging) relative to the tip of the single needle and further relative to one or more anatomical landmarks visible with the ultrasound imaging. For example, with reference to FIG. 2A, a portion of the needle 201 may be visible on ultrasound imaging, but a location of its tip may not be precisely known. After the quantity of saline with microbubbles is injected (408), the microbubbles may be visible on ultrasound, as depicted in FIG. 2B—enabling a clinician to confirm (411) desired location of the tip of the single needle.

The method 400 may include injecting (414), with the second syringe, a therapeutic compound at the injection site. For example, the method 400 may include injecting a therapeutic compound (e.g., by depressing the second-syringe plunger 308, to inject, for example, a corticosteroid 313 from the second syringe 307).

In many instances, the desired location will be relative to specific anatomic landmarks or structures—for example, in a bursa, such as the subacromial bursa 101, illustrated in FIG. 1A, or a joint, such as the glenohumeral join 122, illustrated in FIG. 1B. Such anatomic structures may be visible in ultrasound imaging (e.g., the tissue plane 205 shown in FIG. 2B), and the microbubbles may enable a clinician to confirm (411) location of the single needle relative to such structures.

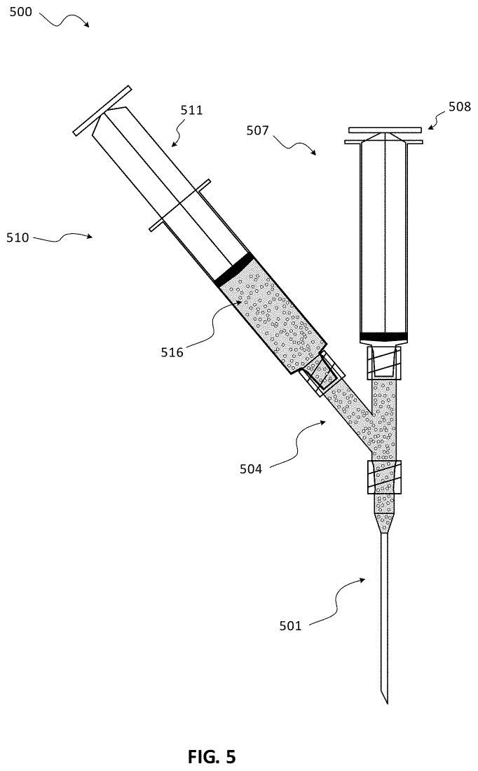

FIG. 5 illustrates an exemplary needle and syringe device 500 that can be used to aspirate a joint. As shown, the device 500 includes a single needle 501, a Y-connector 504 (as shown, with Luer lock connectors), a first syringe 510 having a first-syringe plunger 511, and a second syringe 507 having a second-syringe plunger 508. The first syringe 510 may contain an agitated saline 516 for injecting a small quantity of bubbles to help a medical care provider to place the needle 501; and the second syringe 507 may be configured to aspirate fluid from a treatment site (e.g., the second syringe 507 may be empty, with its corresponding second-syringe plunger 508 fully depressed.

The device 500 illustrated in FIG. 5 is shown having two equally sized syringes coupled by a rigid Y-connector 504, but the device 500 could take other forms. For example, the agitated saline 516 could be coupled to the Y-connector 504 with flexible tubing to make the overall device 500 more easy to manipulate with one hand by a medical care provider who may be both operating the ultrasound transducer and performing an aspiration. The syringes 507 and 510 may be larger or smaller or differently sized. Rather than the needle 501 having a single lumen, it may be a double-lumen needle, and each lumen may be separately connected to a different syringe. Other variations are possible.

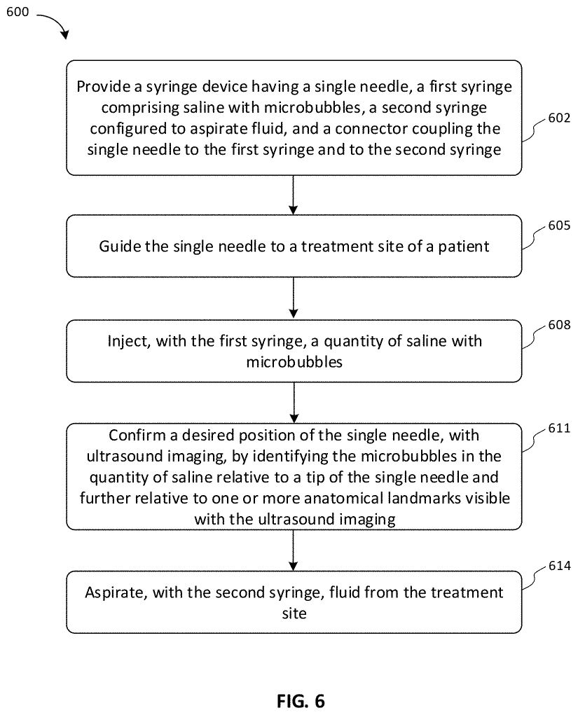

FIG. 6 illustrates an exemplary method 600 for performing an aspiration. As illustrated, the method 600 includes providing (602) a syringe device. In particular, the method 600 includes providing (602) a syringe device having a single needle (e.g., the device 500, shown in FIG. 5, having a single needle 501), a first syringe comprising saline with microbubbles (e.g., syringe 510), a second syringe configured to aspirate fluid (e.g., syringe 507), and a connector coupling the single needle to the first syringe and second syringe (e.g., connector 504).

The method 600 may include guiding (605) the single needle to a treatment site of a patient. For example, with reference to FIG. 1B, the method 600 may include guiding the single needle to the glenohumeral joint of a patient.

The method 600 may include injecting (608), with the first syringe, a quantity of saline with microbubbles. For example, with reference to FIG. 5, the method 600 could include injecting (608), e.g., by depressing the first-syringe plunger 511, to inject a quantity of saline with microbubbles.

The method 600 may include confirming (611) the desired position of the single needle, with ultrasound imaging. Confirming (611) the desired position of the single needle could include identifying microbubbles in the quantity of saline (via ultrasound imaging) relative to the tip of the single needle and further relative to one or more anatomical landmarks visible through the ultrasound imaging. For example, with reference to FIG. 2A, a portion of the needle 201 may be visible on ultrasound imaging, but a location of its tip may not be precisely known. After the quantity of saline with microbubbles is injected (608), the microbubbles may be visible on ultrasound, as depicted in FIG. 2B—enabling a clinician to confirm (611) desired location of the tip of the single needle.

The method 600 may include aspirating (614), with the second syringe, fluid at the treatment site. For example, the method 600 may include aspirating (614) the glenohumeral joint of a patient by drawing back the second-syringe plunger 508, to aspirate the joint (e.g., to facilitate analysis of aspirated synovial fluid, to, for example, diagnose a joint infection).

While several implementations have been described with reference to exemplary aspects, it will be understood by those skilled in the art that various changes may be made and equivalents may be substituted for elements thereof without departing from the contemplated scope. For example, corticosteroids are provided as an example therapeutic compound; but other therapeutic compounds are also contemplated, such as, for example, local anesthetics (e.g., lidocaine, bupivacaine), hyaluronic acid or other viscosupplementation therapies to improve lubrication of joints, or other compounds for providing relief from joint issues or to promote healing of joint, muscle or ligament injuries (e.g., platelet rich plasma, or PRP; stem cells; nerve blocks, etc.).

In addition, many modifications may be made to adapt a particular situation or material to the teachings provided herein without departing from the essential scope thereof. Therefore, it is intended that the scope not be limited to the particular aspects disclosed but include all aspects falling within the scope of the appended claims.

What is claimed is:

1. A method comprising:
providing a syringe device having a single needle, a first syringe comprising saline with microbubbles, a second syringe comprising a therapeutic compound, and a connector coupling the single needle to the first syringe and to the second syringe;
guiding the single needle to an injection site of a patient;
injecting, with the first syringe, a quantity of saline with microbubbles;
confirming a desired position of the single needle, with ultrasound imaging, by identifying the microbubbles in the quantity of saline relative to a tip of the single needle and further relative to one or more anatomical landmarks visible with the ultrasound imaging; and
injecting, with the second syringe, the therapeutic compound at the injection site.

2. The method of claim 1, wherein the therapeutic compound comprises a corticosteroid.

3. The method of claim 1, wherein the therapeutic compound comprises lidocaine.

4. A method comprising:
providing a syringe device having a single needle, a first syringe comprising saline with microbubbles, a second syringe configured to aspirate fluid from a treatment site of a patient, and a connector coupling the single needle to the first syringe and to the second syringe;
guiding the single needle to the treatment site;
injecting, with the first syringe, a quantity of saline with microbubbles;
confirming a desired position of the single needle, with ultrasound imaging, by identifying the microbubbles in the quantity of saline relative to a tip of the single needle and further relative to one or more anatomical landmarks visible with the ultrasound imaging; and
aspirating, with the second syringe, fluid from the treatment site.

5. A method comprising:
providing a syringe device having a single needle, a first syringe comprising saline with microbubbles and having a first-syringe plunger, a second syringe having a second-syringe plunger that is independently operable relative to the first-syringe plunger, and a connector coupling the single needle to the first syringe and to the second syringe;
guiding the single needle to a treatment site of a patient;
injecting, with the first syringe, a quantity of saline with microbubbles;
confirming a desired position of the single needle, with ultrasound imaging, by identifying the microbubbles in the quantity of saline relative to a tip of the single needle and further relative to one or more anatomical landmarks visible with the ultrasound imaging; and
with the second syringe, injecting a therapeutic compound at the treatment site or aspirating fluid from the treatment site.

6. The method of claim 5, wherein injecting with the first syringe comprises depressing the first-syringe plunger.

7. The method of claim 5, wherein the second syringe comprises the therapeutic compound, and wherein injecting the therapeutic compound comprises depressing the second-syringe plunger.

8. The method of claim 5, wherein the second syringe is configured to aspirate fluid from a treatment site of a patient, and wherein aspirating fluid from the treatment site comprises drawing back the second-syringe plunger.
